(19) United States
(12) Patent Application Publication
(10) Pub. No.: US 2022/0305143 A1
(43) Pub. Date: Sep. 29, 2022

(54) COMPOSITIONS AND METHODS OF DETECTING AND TREATING THROMBOSIS AND VASCULAR PLAQUES

(71) Applicant: MICROVASCULAR THERAPEUTICS, LLC, Tucson, AZ (US)

(72) Inventors: Evan C. Unger, Tucson, AZ (US); Emmanuelle Joelle Meuillet, Tucson, AZ (US); Iman Daryaei, Tucson, AZ (US); Maria Fernanda Acosta, Tucson, AZ (US)

(21) Appl. No.: 17/616,166

(22) PCT Filed: Jun. 1, 2020
(86) PCT No.: PCT/US20/35580

§ 371 Date: Dec. 2, 2021

Related U.S. Application Data
(60) Provisional application No. 62/857,766, filed on Jun. 5, 2019.

(51) Int. Cl. A61K 49/22; A61K 41/00; A61K 47/51; A61K 47/26; A61P 7/02; A61K 47/10

(52) U.S. Cl. CPC A61K 49/223; A61K 49/226; A61K 41/0028; A61K 47/51; A61K 47/26; A61P 7/02; A61K 47/10

(57) ABSTRACT

The invention provides nanodroplets labeled with targeting ligands that are useful in the detection and treatment of vascular thromboses (e.g., fibrin clots) and vascular plaques, or related diseases and conditions, as well as methods of preparation and use thereof.

### PRIORITY CLAIMS AND RELATED PATENT APPLICATIONS

This application claims the benefit of priority from U.S. Provisional Application Ser. No. 62/857,766, filed on Jun. 5, 2019, the entire content of which is incorporated herein by reference for all purposes.

### TECHNICAL FIELDS OF THE INVENTION

This invention relates to pharmaceutical compositions and methods of their preparation and diagnostic or therapeutic use. More particularly, the invention relates to targeted microbubbles and/or nanodroplets, and emulsions thereof, labeled with diagnostic and/or therapeutic ligands that are useful in the detection and disruption of vascular thromboses (e.g., fibrin clots) and vascular plaques, as well as methods of preparation and use thereof.

### BACKGROUND OF THE INVENTION

Cardiovascular disease (CVD) is the leading cause of death and disability worldwide. Thrombosis is the underlying cause of many types of CVD, including venous thromboembolism (VTE), ischemic heart disease and ischemic stroke. Efforts to remove occlusive thrombi by angioplasty/stenting, thromboembolectomy, mechanical disruption, and/or biochemical dissolution have had mixed efficacies. These techniques are generally time consuming and costly to perform and are often accompanied by substantial risk of hemorrhagic complications.

Microbubbles have been used to enhance coronary sonothrombolysis in treatment of acute myocardial infarction (MI) and in acute ischemic stroke. In both MI and ischemic stroke, a thrombus causes arterial blockage depriving the tissues downstream of blood flow leading to ischemia and potentially cellular death. Thrombi are composed variably of fibrin and platelets which may be rich in red blood cells enmeshed within.

Fibrin, also called Factor Ia, is a fibrous, non-globular protein involved in the clotting of blood. Fibrin is present at high concentrations in both venous and arterial thrombosis providing high sensitivity to fibrin-targeting therapies. At the same time, fibrin is not present in circulating blood, which allows potentially high specificity for these therapies. Besides protein-based approaches, small cyclic peptides which present high affinity for fibrin and high selectivity over fibrinogen have also been described. The potential benefits of small peptides in comparison to antibodies include faster bloodstream clearance and the ability to penetrate into the fibrin mesh, both of which result in improved target-to-background ratios.

Inflammation and endothelial dysfunction are key threshold developments in the progression of atherosclerosis. Expression of endothelial cell adhesion molecules, e.g., vascular cell adhesion molecule-1 (VCAM-1), has been shown to play an important role in recruitment of leukocytes and is often increased at sites of pathological inflammation. Persistent expression of VCAM-1 in dysfunctional endothelial cells mediates adhesion, rolling, and tethering of mononuclear leukocytes and facilitates their transmigration to developing atherosclerotic plaques. VCAM-1 is thus a target for not only early detection by imaging but also for therapeutic drug delivery.

Ultrasound can be used to disrupt thrombi; however, there is a trade-off between time/efficiency and damage to healthy tissue. Reagents, such as microbubbles, that can locally amplify the sound can accelerate disruption while keeping delivered energy low. A caveat to the use of bubbles stems from their size (1-5 microns), which may prevent access to the thrombus interior. Thrombi present porous matrices but the interstices of the clot generally preclude entry of micron-sized structures.

Thus, there remains an ongoing need for improved therapeutics and methods for detection and treatment of thrombosis and related diseases and conditions. Efforts to enhance safety, efficacy and efficiency of thrombus removal have high potential clinical impact.

### SUMMARY OF THE INVENTION

The invention is based in part on novel microbubbles and nanodroplets with targeting capabilities to select biomarkers and emulsions thereof useful in diagnosis and treatment of certain diseases and conditions, in particular thrombosis. These carriers are capable of targeting various protein targets, such as fibrin and VCAM-1, for improved detection or disruption of thrombus, platelets and vascular plaques occurring in cardiovascular diseases. The invention further relates to pharmaceutical compositions and methods of preparation and use thereof.

In one aspect, the invention generally relates to an aqueous emulsion or suspension of microbubbles and/or nanodroplets having one or more fibrin-binding ligands attached thereto.

In another aspect, the invention generally relates to an aqueous emulsion or suspension of microbubbles and/or nanodroplets having one or more VCAM-1-binding ligands attached thereto.

In yet another aspect, the invention generally relates to an aqueous emulsion or suspension comprising microbubbles and/or nanodroplets having one or more fibrin-binding ligands attached thereto as disclosed herein and microbubbles and/or nanodroplets having one or more VCAM-1-binding ligands attached thereto as disclosed herein.

In yet another aspect, the invention generally relates to a method for detecting a vascular thrombus or plaque. The method comprises: administering to a subject in need thereof an aqueous emulsion or suspension disclosed herein; and imaging a part of the subject to detect the presence of vascular thrombus or plaque.

In yet another aspect, the invention generally relates to a method for diagnosing or assessing thrombosis. The method comprises: administering to a subject in need thereof an aqueous emulsion or suspension disclosed herein; and imaging a part of the subject to diagnose or assess thrombosis in the subject.

In yet another aspect, the invention generally relates to a method for disrupting or destroying vascular thromboses or plaques. The method comprises: administering to a subject in need thereof an aqueous emulsion or suspension disclosed herein; and applying ultrasound to a targeted region of an organ of the subject having vascular thromboses or plaques thereby destroying or reducing the vascular thromboses or plaques.

In yet another aspect, the invention generally relates to a method for treating thrombosis or arterial plaque. The method comprises: administering to a subject in need thereof an aqueous emulsion or suspension disclosed herein; and applying ultrasound to a targeted region of the subject.

In yet another aspect, the invention generally relates to a method for performing sonothrombolysis. The method comprises: administering to a subject in need thereof an aqueous emulsion or suspension disclosed herein; and applying ultrasound to a targeted region of the subject.

### BRIEF DESCRIPTION OF THE DRAWINGS

FIG. 1. A Fibrin Binding Peptide (FBP) with an azide functional group conjugated to DSPE-PEG5000-DBCO to make a product with a dibenzocycoocta triazole linker.

FIG. 2. FBP with an amine functional group conjugated to DSPE-PEG5000-NHS Ester to make a product with an amide linker.

FIG. 3. Perfluorobiphenyl sulfide was oxidized to generate a more active sulfone derivative which was then reacted with DSPE-PEG5000-Amine to produce DSPE-PEG5000-PFPhSO₂. Finally, DSPE-PEG5000-PFPhSO₂ was reacted with FBP bearing an amine group to yield the conjugated final product.

FIG. 4. Conjugation of FBP to DSPE-PEG5000-DBCO (A), DSPE-PEG5000-NHS Ester (B) and DSPE-PEG5000-PFPhSO₂ (C) was confirmed by MS data.

FIG. 5. FBP tagged with 5(6)-carboxytetramethylrhodamine N-succinimidyl ester to produce FBP-Rh (MW=2100.75 Da) (top), and DK-12 tagged with 5(6)-carboxytetramethylrhodamine N-succinimidyl ester to produce DK-12-Rh (MW=2182.49 Da) (bottom).

FIG. 6. In vitro affinity binding assay of fluorescence (Rhodamine label) of control peptide (DK12) vs. fluorescence (Rhodamine label) fibrin-binding peptide.

FIG. 7. A general representation of targeted MBs. MBs in which combination of various phospholipids formed a spherical shell while inside was filled with a perfluorocarbon gas preferentially octafluoropropane. Target binding ligands including VCAM-1 ligand or FBP (shown as green stars) was attached to the surface shell of the bubble via PEG linkers.

FIG. 8. Size distribution of various types of MBs with the different FBP conjugated phospholipids and MPEG control (A) and Number-Weighted average of all samples (B).

FIG. 9. Gas content of MBs. The gas content of all 4 types of samples were measured by GC.

FIG. 10. TEM micrographs of (A) Fibrin binding peptide targeted microbubble; (B) Fibrin binding peptide targeted nanodroplet.

FIG. 11. TEM micrographs of (A) Fibrin binding peptide targeted microbubble permeating a fibrin clot; (B) Fibrin binding peptide targeted nanodroplet permeating a fibrin clot.

FIG. 12. VCAM-1 ligand was conjugated to DSS linker through the N-terminal amine group. DSPE-PEG2K-Amine was conjugated to the other head of DSS linker to results in VCAM-1_DSPE-PEG2K conjugate.

FIG. 13. Exemplary fluorescence data on disruption of fibrin clots.

### DETAILED DESCRIPTION OF THE INVENTION

The invention provides novel microbubbles and nanodroplets with targeting capabilities to select biomarkers, and emulsions thereof, that are useful as diagnostic probes and therapeutic agents for certain diseases and conditions, in particular thrombosis and arterial plaques. These microbubbles and/or nanodroplets are capable of targeting various protein targets, such as fibrin and VCAM-1, for improved detection and/or disruption of blood clots (e.g., thrombus, platelets and vascular plaques) occurring in a number of cardiovascular diseases. The targeting microbubbles and/or nanodroplets may be acoustically activated in situ to cause blood clots disruption. The invention further provides pharmaceutical compositions and methods of preparation and use thereof.

A key feature of the present invention is the nanoscale, acoustically active nanodroplets, e.g., in the range from about 100 nm to about 300 nm, which is a fraction of the size of typically microbubbles. The smaller sizes allow the droplets to more easily penetrate the thrombus and thus significantly increase the sonothrombolytic efficiency and clinical efficacy.

Another key feature of the invention is that low temperature and high pressure is used to condense fluorocarbon microbubbles (e.g., octafluoropropane microbubbles) into nanodroplets (e.g., octafluoropropane nanodroplets). Even though the boiling point (−34° C.) of octafluoropropane is substantially below body temperature, the nanodroplets stay condensed after Intravenous (IV) administration and then reform microbubbles after they enter the acoustic field.

Yet another key feature of the invention is that the nanodroplets, which bear one or more targeting ligands, can be acoustically and locally activated in situ. High specificity can be achieved as fibrin is not present in circulating blood. Small peptides employed as targeting ligands herein exhibit high affinity for fibrin and high selectivity over fibrinogen. These small peptides provide the advantage of faster bloodstream clearance and the ability to penetrate into the fibrin mesh, leading to improved target-to-background ratios.

Yet another key feature of the invention is the unique formulation disclosed here, which provides the nanodroplets with enhanced sufficient stability required for manipulation and handling during preparation, storage and treatment procedures.

Disclosures of U.S. Pat. No. 9,801,959 B2 and PCT/US19/24713, filed Mar. 28, 2019 are incorporated herein by reference in their entireties for all purposes.

In one aspect, the invention generally relates to an aqueous emulsion or suspension of microbubbles and/or nanodroplets having one or more fibrin-binding ligands attached thereto.

In certain embodiments, each of microbubbles and/or nanodroplets is conjugated to a plurality of the fibrin-binding ligands.

In certain embodiments, the one or more fibrin-binding ligands comprise fibrin-binding peptides having from about 11 to about 16 amino acids.

In certain embodiments, the fibrin-binding peptides are selected from: Tn6, Tn7, or Tn10 families (Table 1)

TABLE 1
Examples of Fibrin-Specific Peptides

| Tn6 family | Tn7 family | Tn10 family |
|---|---|---|
| 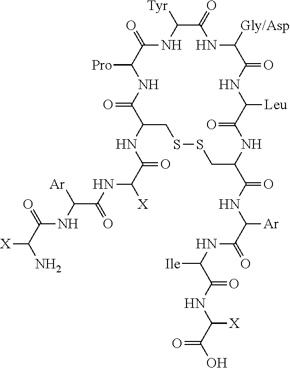 | 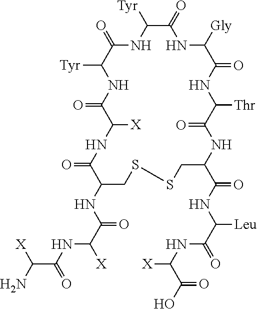 | 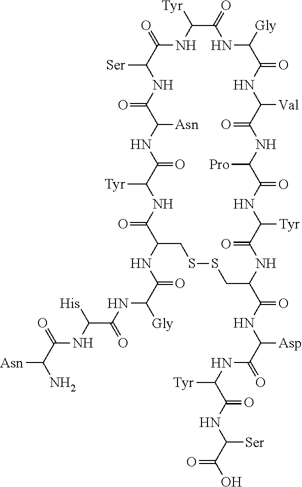 |

Oliveira et al. 2017 Dalton Trans. 46 (42): 14488-14508.
Kolodziej, et al. 2012 Bioconj. Chem. 23:548-556.

In certain embodiments, the fibrin-binding ligands are conjugated to the microbubbles and/or nanodroplets via a bi-functional spacer, preferably a polyethylene glycol (PEG) group, preferably having a number average molecular weight (MW) in the rage from about 1,000 to about 10,000 Daltons (e.g., from about 2,000 to about 10,000, from about 3,000 to about 10,000 Daltons, from about 4,000 to about 10,000 Daltons, from about 1,000 to about 8,000 Daltons, from about 1,000 to about 6,000 Daltons, from about 3,000 to about 7,000 Daltons, from about 4,000 to about 6,000 Daltons) and more preferably about 5,000 Daltons. The PEG group is covalently bound to a lipid anchor, preferably a phospholipid.

In certain embodiments, the phospholipid composition comprises dipalmitoylphosphatidylcholine (“DPPC”). DPPC is a zwitterionic compound, and a substantially neutral phospholipid. In certain embodiments, the composition comprises a PEG'ylated lipid.

Examples of lipids include phosphoethanolamine-N-[methoxy(polyethylene glycol)-2000] (ammonium salt), 1,2-distearoyl-sn-glycero-3-phosphoethanolamine-N-[methoxy(polyethylene glycol)-2000] (ammonium salt), 1,2-dioleoyl-sn-glycero-3-phosphoethanolamine-N-[methoxy(polyethylene glycol)-2000] (ammonium salt), 1,2-dimyristoyl-sn-glycero-3-phosphoethanolamine-N-[methoxy(polyethylene glycol)-3000] (ammonium salt), 1,2-dipalmitoyl-sn-glycero-3-phosphoethanolamine-N-[methoxy(polyethylene glycol)-3000] (ammonium salt), 1,2-distearoyl-sn-glycero-3-phosphoethanolamine-N-[methoxy(polyethylene glycol)-3000] (ammonium salt), 1,2-dioleoyl-sn-glycero-3-phosphoethanolamine-N-[methoxy(polyethylene glycol)-3000] (ammonium salt), 1,2-dimyristoyl-sn-glycero-3-phosphoethanolamine-N-[methoxy(polyethylene glycol)-5000] (ammonium salt), 1,2-dipalmitoyl-sn-glycero-3-phosphoethanolamine-N-[methoxy(polyethylene glycol)-5000] (ammonium salt), 1,2-distearoyl-sn-glycero-3-phosphoethanolamine-N-[methoxy(polyethylene glycol)-5000] (ammonium salt) and 1,2-dioleoyl-sn-glycero-3-phosphoethanolamine-N-[methoxy(polyethylene glycol)-5000] (ammonium salt). Dipalmitoylphosphatidylethanolamine (“DPPE”) is a preferred lipid, preferably in the formulation with the other lipids at concentration of between 5 and 20 mole percent, most preferably 10 mole percent.

In certain embodiments, the microbubbles and/or nanodroplets are filled with a gaseous material.

In certain embodiments, the gaseous material comprises a fluorinated gas. The term “fluorinated gas”, as used herein, refers to hydrofluorocarbons, which contain hydrogen, fluorine and carbons, or to compounds which contain only carbon and fluorine atoms (also known as perfluorocarbons) and to compounds containing sulfur and fluorine. In the context of the present invention, the term may refer to materials that are comprised of carbon and fluorine or sulfur and fluorine in their molecular structure and are gases at normal temperature and pressure.

In certain embodiments, the fluorinated gas is selected from perfluoromethane, perfluoroethane, perfluoropropane, perfluorocyclopropane, perfluorobutane, perfluorocyclobutane, perfluoropentane, perfluorocylcopentane, perfluorohexane, perfluorocyclohexane, and mixtures of two or more thereof.

In certain embodiments, the fluorinated gas is selected from perfluoropropane, perfluorocyclopropane, perfluorobutane, perfluorocyclobutane, perfluoropentane, perfluorocylcopentane, and mixtures of two or more thereof.

In certain embodiments, the fluorinated gas comprises octafluoropropane.

In certain embodiments, the aqueous emulsion or suspension further comprises a stabilizing agent.

In certain embodiments, the stabilizing agent is selected from the group consisting of D (+) trehalose dihydrate, propylene glycol, glycerol, polyethylene glycol, glucose and sucrose.

In certain embodiments, the gaseous material further comprises a suitable percentage of non-fluorinated gas or gas mixture, for example, about 2% to about 20% air or nitrogen (e.g., from about 5% to about 20%, from about 10% to about 20%, from about 15% to about 20%, from about 2% to about 15%, from about 2% to about 10%, from about 2% to about 5% of air or nitrogen).

In certain embodiments the fluorocarbon within the microbubbles and/or nanodroplets exist in a condensed, i.e. liquid state.

In another aspect, the invention generally relates to an aqueous emulsion or suspension of microbubbles and/or nanodroplets having one or more VCAM-1-binding ligands attached thereto.

In certain embodiments, each of microbubbles and/or nanodroplets is conjugated to a plurality of the VCAM-1-binding ligands.

In certain embodiments, the one or more VCAM-1-binding ligands are VCAM-1-binding peptides having from about 8 to about 16 amino acids.

In certain embodiments, the VCAM-1-binding peptides are selected from: B2702p1-20 Peptides (Table 2).

TABLE 2
Exemplary VCAM-1-binding Peptides

| Name | Peptide Sequence |
|---|---|
| B2702p | HGR ENL RIA LRY |
| B2702p1 | HGR ANL JUL ARY |
| B2702p2 | HGR ENL AIL ARY |
| B2702p3 | HGR ENL JUL ARA |
| B2702p4 | HGR ENL JUL AAY |
| B2702p5 | HGR ENL JUL ARY |
| B2702p6 | HGR ENA JUL ARY |
| B2702p7 | HGA ENL JUL ARY |
| B2702p8 | HGR ENL RIA ARY |
| B2702p9 | HGR EAL JUL ARY |
| B2702p10 | HGR ENL JUL ARY |
| B2702p11 | HGA ENL RIA LRY |
| B2702p12 | HGR ANL RIA LRY |
| B2702p13 | HGR EAL RIA LRY |
| B2702p14 | HGR ENA RIA LRY |
| B2702p15 | HGR ENL AIA LRY |
| B2702p16 | HGR ENL RAA LRY |
| B2702p17 | HGR ENL RIA LAY |
| B2702p18 | HGR ENL RIA LRA |
| B2702p19 | HGR ANL JUL ARA |
| B2702p20 | HGR ANL JUL AAY |

Dimastromatteo, et al. 2013 J Nucl Med. 54(8):1442-9.

In certain embodiments, the VCAM-1-binding ligands are conjugated to the microbubbles and/or nanodroplets via a PEG linker disclosed herein.

In certain embodiments, the microbubbles and/or nanodroplets are filled with a gaseous material.

In certain embodiments, the gaseous material comprises a fluorinated gas.

In certain embodiments, the fluorinated gas is selected from perfluoromethane, perfluoroethane, perfluoropropane, perfluorocyclopropane, perfluorobutane, perfluorocyclobutane, perfluoropentane, perfluorocylcopentane, perfluorohexane, perfluorocyclohexane, and mixtures of two or more thereof.

In certain embodiments, the fluorinated gas is selected from perfluoropropane, perfluorocyclopropane, perfluorobutane, perfluorocyclobutane, perfluoropentane, perfluorocylcopentane, and mixtures of two or more thereof.

In certain embodiments, the fluorinated gas comprises octafluoropropane.

In certain embodiments, the aqueous emulsion or suspension further comprises a stabilizing agent.

In certain embodiments, the stabilizing agent is selected from the group consisting of D (+) trehalose dihydrate, propylene glycol, glycerol, polyethylene glycol, glucose and sucrose.

In yet another aspect, the invention generally relates to an aqueous emulsion or suspension comprising microbubbles and/or nanodroplets having one or more fibrin-binding ligands attached thereto as disclosed herein and microbubbles and/or nanodroplets having one or more VCAM-1-binding ligands attached thereto as disclosed herein.

In certain embodiments of the aqueous emulsion or suspension disclosed herein, the microbubbles and/or nanodroplets are coated by a film-forming material.

In certain embodiments, the film-forming material comprises one or more lipids.

In certain embodiments, the lipids comprise a phospholipid or a mixture of phospholipids.

Any suitable lipids may be utilized. The lipid chains of the lipids may vary from about 10 to about 24 (e.g., from about 10 to about 20, from about 10 to about 18, from about 12 to about 20, from about 14 to about 20, from about 16 to about 20, 10, 11, 12, 13, 14, 15, 16, 17, 18, 19, 20, 21, 22, 23, 24) carbons in length. More preferably, the chain lengths are from about 16 to about 18 carbons.

In some embodiments, the microscopic or nanoscopic bubble has a diameter in the range of about 10 nm to about 10 μm (e.g., from about 10 nm to about 5 μm, from about 10 nm to about 1 μm, from about 10 nm to about 500 nm, from about 10 nm to about 100 nm, from about 50 nm to about 10 μm, from about 100 nm to about 10 μm, from about 1 μm to about 10 μm). In some embodiments, the microscopic or nanoscopic particle or bubble has a diameter from about 10 nm to about 100 nm. In some embodiments, the microscopic or nanoscopic particle or bubble has a diameter from about 100 nm to about 1 μm. In some embodiments, the microscopic or nanoscopic particle or bubble has a diameter from about 1 μm to about 10 μm.

In certain embodiments, the microbubbles and/or nanodroplets are microbubbles having a microscopic size ranging from about 0.5 to about 10 microns (e.g., from about 1 μm to about 10 μm, from about 2 μm to about 10 μm, from about 5 μm to about 10 μm, from about 0.5 μm to about 5 μm, from about 0.5 μm to about 2 μm, from about 1 μm to about 5 μm).

In certain embodiments, the microbubbles and/or nanodroplets are nanodroplets having a nanoscopic size ranging from about 100 nm to about 800 nm (e.g., from about 100 nm to about 500 nm, from about 100 nm to about 300 nm, from about 120 nm to about 280 nm). In certain embodiments, the microbubbles and/or nanodroplets are nanodroplets having a nanoscopic size ranging from about 120 nm to about 280 nm.

In certain embodiments, the microbubbles and/or nanodroplets do not comprise microbubbles and/or nanodroplets having a size outside of about 120 nm to about 280 nm (i.e., substantially all microbubbles and/or nanodroplets ate nanodroplets having a nanoscopic size ranging from about 120 nm to about 280 nm).

In certain embodiments, the aqueous emulsion or suspension is in a homogenized form.

In certain embodiments, the aqueous emulsion or suspension further comprises a pharmaceutically acceptable excipient, carrier, or diluent.

In yet another aspect, the invention generally relates to a method for detecting a vascular thrombus or plaque. The method comprises: administering to a subject in need thereof an aqueous emulsion or suspension disclosed herein; and imaging a part of the subject to detect the presence of vascular thrombus or plaque.

In yet another aspect, the invention generally relates to a method for diagnosing or assessing thrombosis or atherosclerosis. The method comprises: administering to a subject in need thereof an aqueous emulsion or suspension disclosed herein; and imaging a part of the subject to diagnose or assess thrombosis in the subject.

In yet another aspect, the invention generally relates to a method for disrupting or destroying vascular thromboses or plaques. The method comprises: administering to a subject in need thereof an aqueous emulsion or suspension disclosed herein; and applying ultrasound to a targeted region of an organ of the subject having vascular thromboses or plaques thereby destroying or reducing the vascular thromboses or plaques.

In yet another aspect, the invention generally relates to a method for treating thrombosis, atherosclerosis or arterial plaque. The method comprises: administering to a subject in need thereof an aqueous emulsion or suspension disclosed herein; and applying ultrasound to a targeted region of the subject.

In yet another aspect, the invention generally relates to a method for performing sonothrombolysis. The method comprises: administering to a subject in need thereof an aqueous emulsion or suspension disclosed herein; and applying ultrasound to a targeted region of the subject.

In certain embodiments of the methods, the fluorinated gas comprises perfluoromethane, perfluoroethane, perfluoropropane, perfluorocyclopropane, perfluorobutane, perfluorocyclobutane, perfluoropentane, perfluorocylcopentane, perfluorohexane, perfluorocyclohexane, and mixtures of two or more thereof.

In certain embodiments of the methods, the fluorinated gas comprises octafluoropropane.

In certain embodiments of the methods, the microbubbles and/or nanodroplets are microbubbles having a microscopic size ranging from about 0.5 to about 10 microns.

In certain embodiments of the methods, the microbubbles and/or nanodroplets are nanodroplets having a nanoscopic size ranging from about 120 nm to about 280 nm.

In certain embodiments of the methods, the microbubbles and/or nanodroplets do not comprise microbubbles and/or nanodroplets having a size outside of about 120 nm to about 280 nm (i.e., substantially all microbubbles and/or nanodroplets ate nanodroplets having a nanoscopic size ranging from about 120 nm to about 280 nm).

As used herein, an “emulsion” refers to a heterogeneous system consisting of at least one immiscible liquid dispersed in another in the form of droplets that may vary in size from nanometers to microns. The stability of emulsions varies widely and the time for an emulsion to separate can be from seconds to years. Suspensions may consist of a solid particle or liquid droplet in a bulk liquid phase. As an example, an emulsion of dodecafluoropentane can be prepared with phospholipid or fluorosurfactant and the conjugate incorporated into the emulsion at a ratio of from about 0.1 mole percent to about 1 mole percent or even as much as 5 mole percent, relative to the surfactant used in stabilizing the emulsion.

In certain embodiments, the emulsion or suspension further comprises a pharmaceutically acceptable excipient, carrier, or diluent. Each excipient, carrier, or diluent must be “acceptable” in the sense of being compatible with the other ingredients of the emulsion or suspension and not injurious to the patient. Some examples of materials which can serve as pharmaceutically acceptable excipient, carrier, or diluent include but not limited to normal saline, phosphate buffered saline, propylene glycol, glycerol and polyethylene glycol, e.g. PEG 400 or PEG 3350 MW.

As used herein, the terms “subject” and “patient” are used interchangeably herein to refer to a living animal (human or non-human). The subject may be a mammal. The terms “mammal” or “mammalian” refer to any animal within the taxonomic classification mammalia. A mammal may be a human or a non-human mammal, for example, dogs, cats, pigs, cows, sheep, goats, horses, rats, and mice. The term “subject” does not preclude individuals that are entirely normal with respect to a disease or condition, or normal in all respects.

As used herein, the terms “treatment” or “treating” a disease or disorder refers to a method of reducing, delaying or ameliorating such a condition, or one or more symptoms of such disease or condition, before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

### EXAMPLES

### Example 1. Preparation of Fibrin-Targeted Bioconjugates

Three conjugation strategies were employed to produce peptide-phospholipid conjugated molecules with different linkers. (1) A fibrin binding peptide (FBP) with a mini-PEG linker and an azide functional group was directly conjugated to N-[dibenzocycooctyl(polyethylene glycol-5000)] carbamyl-distearoylphosphatidyl-ethanolamine (ammonium salt) (DSPE-PEG5000-DBCO) to produce a product with a dibenzocycoocta triazole linker (Scheme 1). (2) An FBP with a mini-PEG linker and amine functional group was conjugated to [(succinimidyloxyglutaryl)aminopropyl, polyethyleneglycol-5000]-carbamyl distearoylphosphatidyl-ethanolamine (sodium salt) (DSPE-PEG5000-NHS ester) to synthesize a product with an amide linker (Scheme 2). (3) The third strategy consisted of first reaction of N-[aminopropyl (polyethyleneglycol-5000)]-carbamyl-distearoylphosphatidyl-ethanolamine (sodium salt) (DSPE-PEG5000-Amine) and 6,6′-sulfonylbis(1,2,3,4,5-pentafluorobenzene) (PFPhSO₂) to produce DSPE-PEG5000-PFPhSO₂. Then the FBP with a mini-PEG linker and amine conjugated with DSPE-PEG5000-PFPhSO₂ to make a product with a perfluorobenzene linker (Scheme 3).

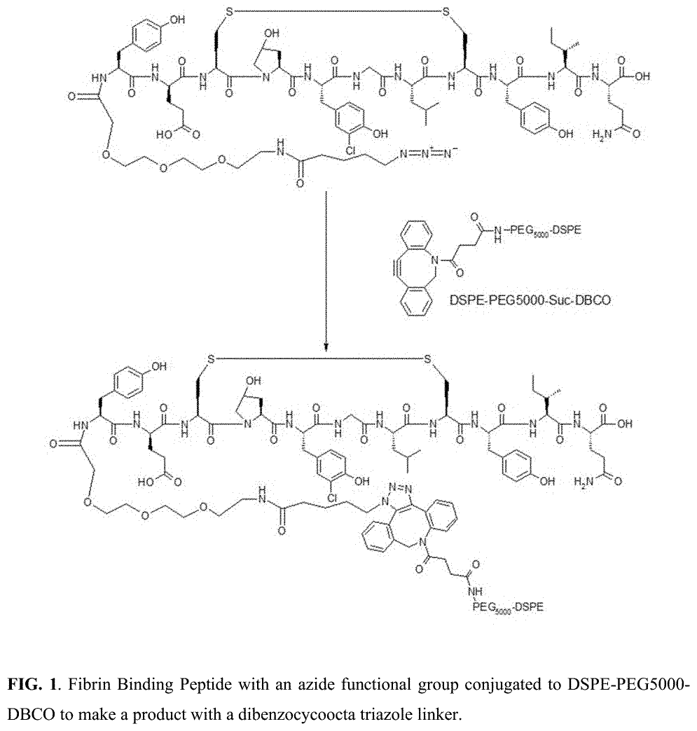

FIG. 1 shows FBP with an azide functional group conjugated to DSPE-PEG5000-DBCO to make a product with a dibenzocycoocta triazole linker.

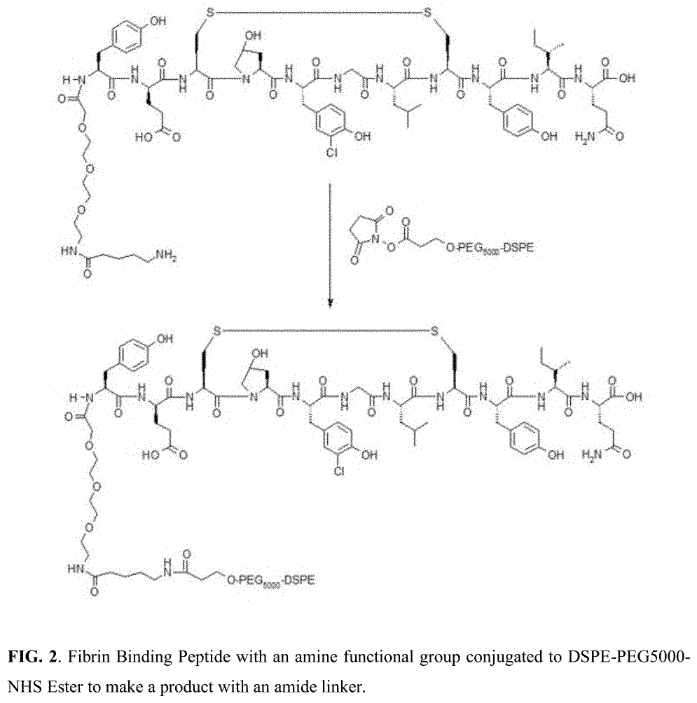

FIG. 2 shows FBP with an amine functional group conjugated to DSPE-PEG5000-NHS Ester to make a product with an amide linker.

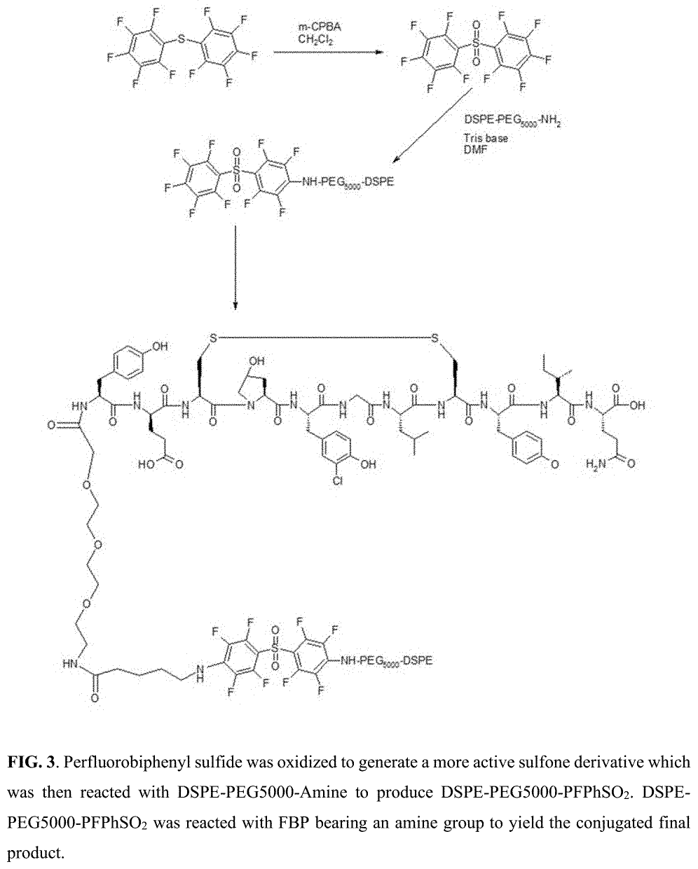

FIG. 3 shows perfluorobiphenyl sulfide was oxidized to generate a more active sulfone derivative which was then reacted with DSPE-PEG5000-Amine to produce DSPE-PEG5000-PFPhSO₂. Finally, DSPE-PEG5000-PFPhSO₂ was reacted with FBP bearing an amine group to yield the conjugated final product.

All products were purified with High-Pressure Liquid Chromatography (HPLC) and characterized with a Mass Spectroscopy (MS) instrument (FIG. 1).

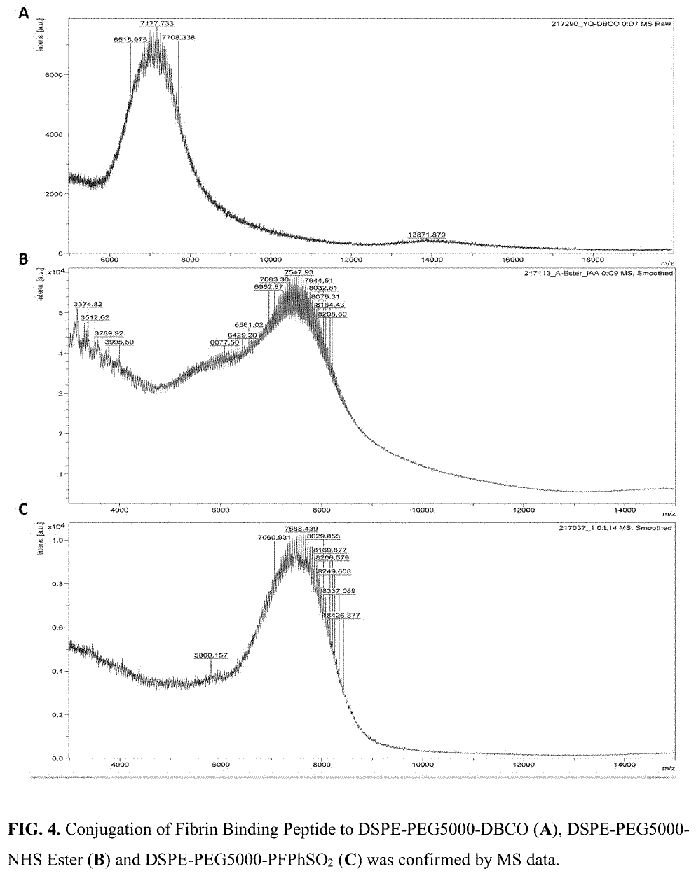

FIG. 4 shows conjugation of FBP to DSPE-PEG5000-DBCO (A), DSPE-PEG5000-NHS Ester (B) and DSPE-PEG5000-PFPhSO₂ (C) was confirmed by MS data.

### Example 2. Fibrin Targeted and Non-Targeted Microbubble Formulation

A mixture of Dipalmitoylphosphatidylcholine (DPPC), 1,2-dipalmitoyl-sn-glycero-3-phosphorylethanolamine (DPPE), N-(Carbonyl-methoxypolyethyleneglycol 5000)-1,2-distearoyl-sn-glycero-3-phosphoethanolamine, sodium salt (DPPE-MPEG5000), and DSPE-PEG5000-FBP conjugates were used in the formulation of targeted microbubbles (MBs) (FIG. 2). DSPE-PEG5000-FBP was replaced with N-(Carbonyl-methoxypolyethyleneglycol 5000)-carbamyl distearoylphosphatidyl-ethanolamine (sodium salt) (DSPE-MPEG5000) in the formulation of non-targeted microbubbles. Vials containing conjugated phospholipid with amide, dibenzocycoocta triazole, and perfluorobenzene linker were named Ester, DBCO, and PFPhSO₂, respectively. Control samples containing DSPE-MPEG5000 were named MPEG for experiments.

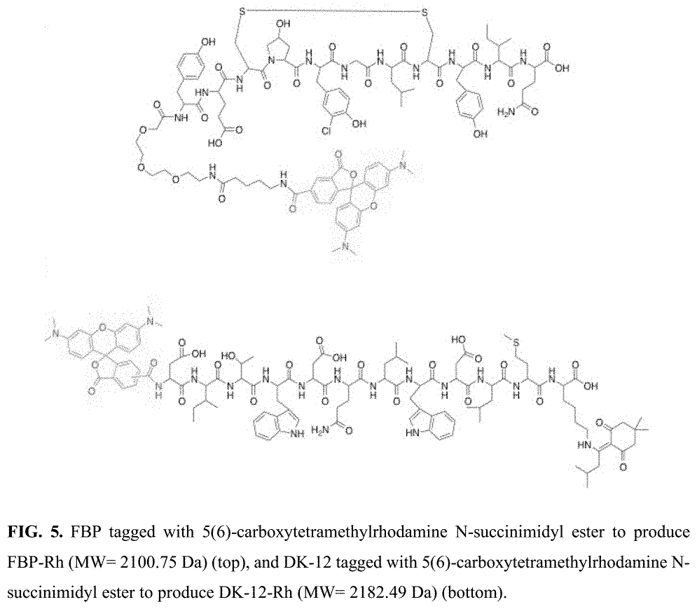

FIG. 5 shows a schematic illustration of targeted MBs in which combination of various phospholipids formed a spherical shell while inside was filled with a perfluorocarbon gas preferentially octafluoropropane. FBP (shown as green stars) was attached to the surface shell of the bubble via PEG linkers.

All vials containing mixture of phospholipids in a solution were filled with octafluoropropane gas (OFP). 2-4 samples of each series of vials were tested for size measurements by a NiComp Acusazie 780 instrument (FIG. 3). Our results showed that all Ester, DBCO, PFPhSO₂, and MPEG samples formed MBs; however, size distribution varied for MBs consisted of different FBP conjugated products. Vials with DBCO and PFPhSO₂ samples showed ˜10% less population of bubbles with a diameter of 0.56-1.06 μm compared to Ester and MPEG vials. In contrast, the DBCO and PFPhSO₂ samples showed over ˜7% and ˜2% more population of bubbles with diameters of 1.06-2.03 and 2.03-5.99 μm, respectively, compared to Ester and MPEG vials (FIG. 3A). No significant difference in the Number-Weighted average of different samples were observed (FIG. 3B).

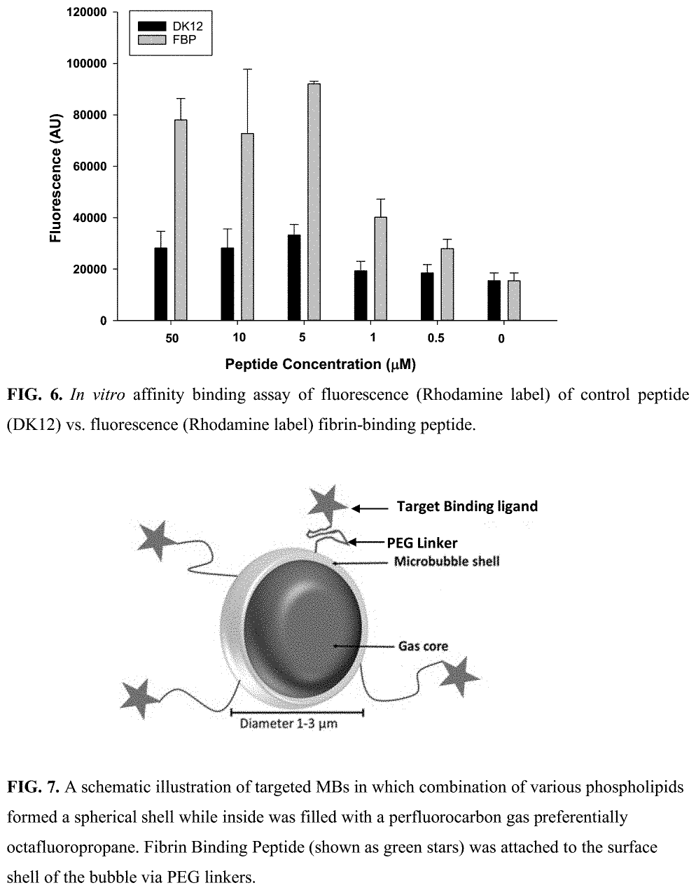

FIG. 6 shows size distribution of various types of MBs with the different FBP conjugated phospholipids and MPEG control (A) and Number-Weighted average of all samples (B). The gas content of each series of vials were analyzed using 2-4 samples from each group by a GC instrument (FIG. 4).

FIG. 7 shows the gas content of all 4 types of samples were measured by GC. Ester samples showed the largest parentage of gas content in this experiment while PFPHSO₂ and MPEG vials showed the lowest amount of the OFP gas. However, GC results confirmed that the gas filling process resulted in gas content >80%, which is very efficient for formation of MBs.

### Example 3. Preparation of VCAM-1-Targeted Bioconjugates

The bioconjugate was prepared by activation of the VCAM-1 ligand in presence of Diisopropylamine and Dimethylformamide. The activated peptide then was reacted with DSPE-PEG5000-NH₂ to form the final product, which was purified by HPLC.

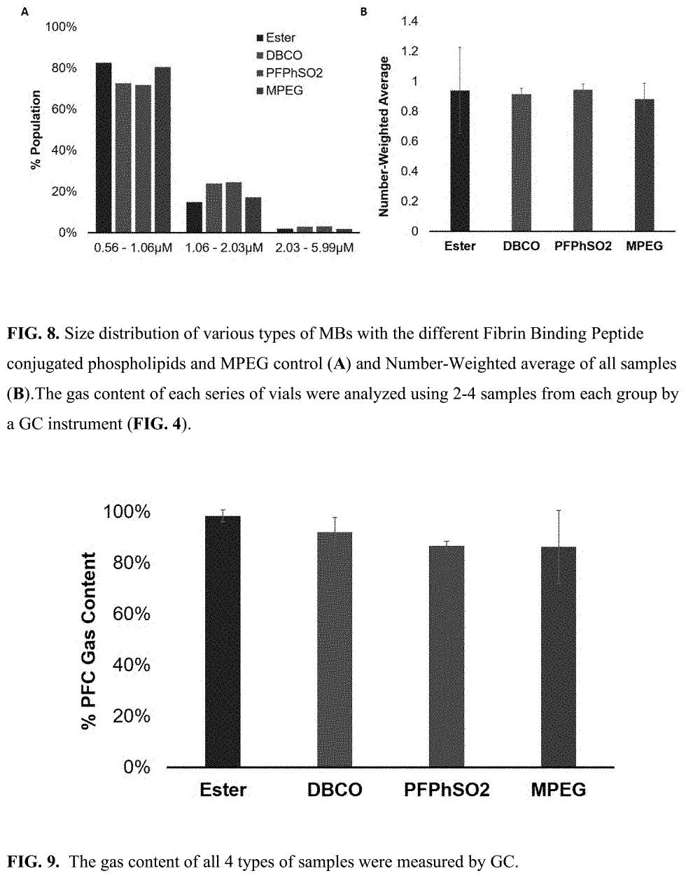

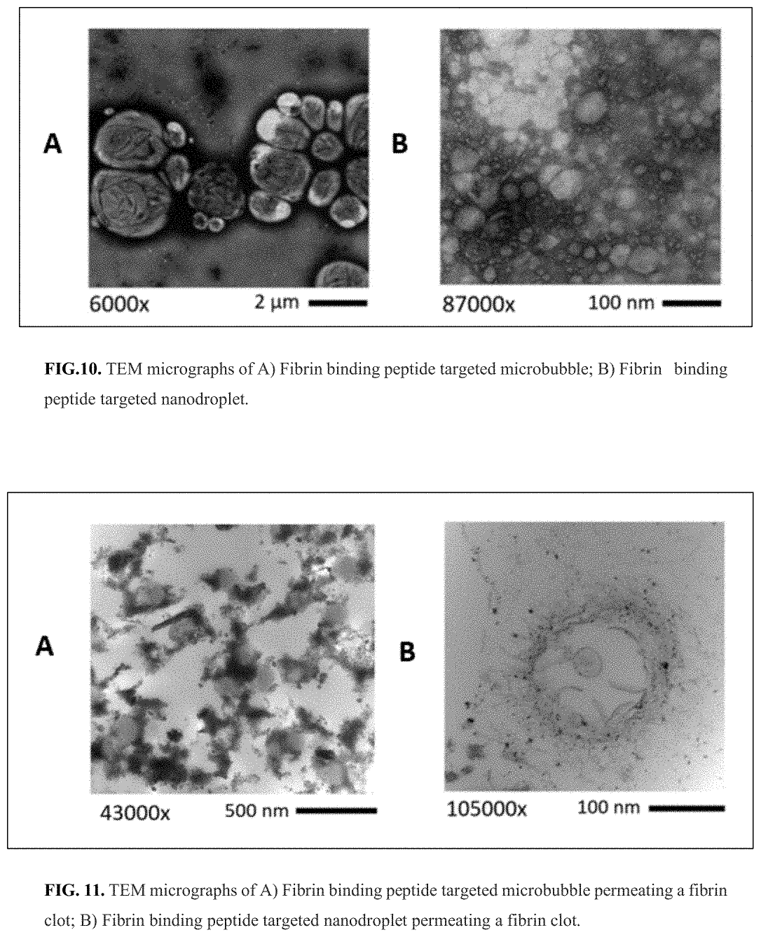

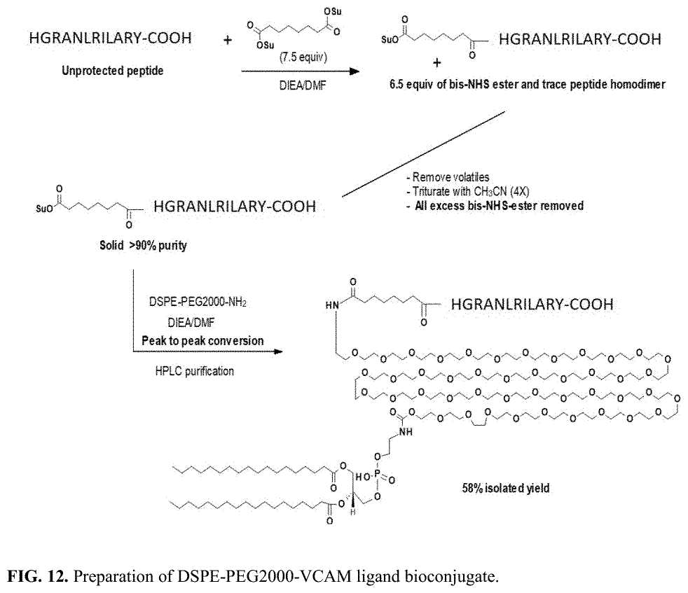

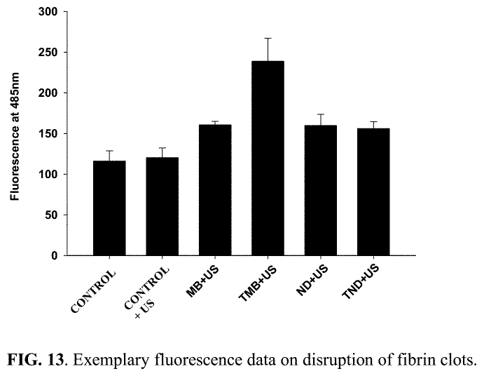

FIG. 8 shows preparation of DSPE-PEG2000-VCAM Ligand bioconjugate.

### Example 4. VCAM-1 Targeted Microbubble Formulation

The targeted microbubble formulation contained dipalmitoylphosphatidylcholine (DPPC), dipalmitoyl-sn-glycerophosphatidylethanolamine-polyethyleneglycol-2000-OMe (DPPE-MPEG-2000) and a lipid-ligand bioconjugate comprised of either DPPE-PEG2000-NH-linked to the ligand via a suberoyl linker (Sub) or DPPE-PEG2000-C(═O)-ligand linked via an amide bond. The conjugates were used at about 1 mol % of the total phospholipids. The microbubbles were prepared by addition of DPPC (90 mol %), DPPE-PEG2000 (9 mol %) and the targeted phospholipid-PEG2000-linker-peptide conjugate (1%) to stirred propylene-glycol at 50-65° C. until the solids were completely dissolved. The warm solution of phospholipids in propylene glycol was then added in several aliquots to a solution of phosphate buffered saline containing 5% glycerol by volume with stirring at 50-65° C.; this solution was stirred 5-10 minutes. The solution was then transferred to a serum vial, which was immediately stoppered, and crimp capped. The solution was allowed to come to ambient temperature and then stored at 4° C. A tranche of 25-50 2 mL nominal capacity serum vials were filled with 1.5 mL aliquots of the chilled phospholipid solution followed by application of light vacuum and purging with perfluorobutane gas followed by rapid stoppering and crimp capping of the vial. Vials were stored at 4° C. until use, whereupon they were allowed to warm to ambient temperature and agitated on a Bristol Myers Squibb Vial Mix apparatus for 45 sec at 75 Hz (4500 rpm) to form the microbubbles.

### Example 5. Preparation of Nanodroplets

Lipid suspensions were prepared from a mixture of DPPC (82%), DPPE (10%), DPPE-MPEG5000 (7%) and DSPE-MPEG5000-FBP bioconjugate (1%) at a total lipid concentration of 0.75 mg/mL in propylene glycol (10.35 mg/mL) by heating at 75° C. for 1 hour. The lipid suspensions were mixed with aqueous solution of Sodium Chloride (4.78 mg/mL), Sodium Phosphate Monobasic (2.34 mg/mL), Sodium Phosphate Dibasic (2.16 mg/mL) and glycerol (12.62 mg/mL) to make the final solution. The final solution was used to fill vials (1.5 mL/vial) and perfluoropropane gas was added to the vials before they were sealed and crimped. Vials incubated for 3 minutes in an ice bath at −15 to −18° C. In addition to the aforementioned excipients the 3% w/v glucose, 0.25% w/v, 0.5% w/v and 1.0% w/v D (+) trehalose dihydrate were also added as excipients. The vials were subjected to agitation for 45 seconds using an amalgam shaker apparatus (Vialmix, BMS Medical Imaging Inc, 4500 rpm) to form a milky appearance, which indicated formation of microbubbles (MBs). The vials were incubated for 3 minutes in an ice bath at −15 to −18° C. The vials were then pressurized at 40-80 psi with N₂ to form a more transparent appearance indicating formation of nanodroplets (NDs). The vials were then incubated for 10 minutes in an ice bath at −15 to −18° C. The vials were kept at room temperature for 1 hour and then were stored at different conditions.

The microbubble referred to as MVT-100 was used as a comparator. All samples were subjected to particle sizing with an AccuSizer 780 (PSS.NiComp Particle Sizing Systems) and a Nanobrook 90 Plus (Brookhaven) size analyzers to measure MB and ND sizes, respectively. The mean size of MVT-100 MB and fibrin-targeted MBs were 1-3 microns. The results are shown in the Table below. The mean size of MVT-100 derived nanodroplets increased rapidly and then decreased as the perfluoropropane gas was lost from the nanodroplets. 3% glucose had a protective effect but not as much as D (+) trehalose dihydrate. 1% D (+) trehalose dihydrate was preferred as this resulted in nanodroplets that were stable for 24 hours.

### Example 6. Disruption of Fibrin Clots by the FTMB

All of the wells of the 24-well plate were coated with fibrin by adding fibrinogen and thrombin and allowing the plates to sit overnight. Briefly, 160 μL of Fibrinogen (1.75 μM in PBS) was added to each well in the presence of 30 μM Thioflavin. Thrombin (40 μL of 7.5 Units/mL in PBS) was added to each well subsequently. The plates were incubated at room temperature overnight in a dark space. Fibrin clots were visualized under a contrast phase microscope.

TABLE 3
Stability of Different Nanodroplet Formulations Incubated at 37° C. (n = 3)

| Formulation | After 1 hour | After 3 hours | After 24 hours |
|---|---|---|---|
| MVT-100 | 1260.04 ± 869.45 | 2602.7 ± 1607.78 | 199.1 ± 82.97 |
| MVT-100-3% | 1001.60 ± 193.34 | 1120.5 ± 161.64 | 5942.22 ± 5746.52 |
| glucose | | | |
| MVT-100- | 3697.97 ± 5073.78 | 1492.65 ± 200.856 | 2008.51 ± 497.56 |
| 0.25% | | | |
| trehalose | | | |
| MVT-100- | 743.27 ± 497.49 | 749.07 ± 490.02 | 3826.81 ± 5820.28 |
| 0.5% | | | |
| trehalose | | | |
| MVT-100-1% | 249.985 ± 12.47 | 241.02 ± 4.64 | 285.11 6.85 |
| trehalose | | | |

TABLE 4
Size distribution, gas content, and zeta potential of control, naked and targeted with FBP
microbubbles and nanodroplets (n = 3).

| | | | | | Zeta |
|---|---|---|---|---|---|
| | | Particle size | OFP (%) in | Concentration | Potential |
| Formulation | | (nm) | headspace | of OFP (mg/mL) | (mV) |
| Control | MB | 776.00 ± 30.00 | 94.47 ± 3.24 | 7.36 ± 0.25 | 0.41 |
| | ND | 213.00 ± 14.99 | | | |
| Naked | MB | 880.00 ± 11.11 | 88.28 ± 2.57 | 7.24 ± 0.21 | −0.234 |
| | ND | 245.51 ± 42.05 | | | |
| Targeted (FBP) | MB | 820.00 ± 34.00 | 86.34 ± 3.91 | 6.73 ± 0.30 | Not available |
| | ND | 149.91 ± 33.15 | | | |

MB were activated (Vial Mix agitation, 45 seconds). The final stock solution of each MB formulation was made with 500 μL in 5.2 mL PBS. The fibrin coated wells are washed with PBS (1.0 mL×1) prior to the addition of MB to the wells. MB were incubated for 3 min. in the fibrin coated wells.

Ultrasound were delivered in each well for a 30 s period (parameters: 2000 mW, PRF 10, 10 ms burst length, frequency 590 Hz).

Supernatant were collected and spun down at 10000 rpm during 15 min. at room temperature. Released fluorescence was measured in a dark 96-well plate. Fluorescence of Thioflavin was measured at 485 nm (λexcit=450 nm; λemis=485 nm).

In one example the readout of the power level on the amplifier was 2,000 mW but the power reading on the wattmeter in line with the transducer was about 100 mW. The estimated mechanical index of the ultrasound was about 0.28 Megapascals (FIG. 9).

In another example, MI of the ultrasound greater than 0.40 Megapascals is used in sonothrombolysis for the ND.

### Example 8

A patient with acute STEMI is treated with nanodroplet enhanced sonothrombolysis. The nanodroplet formulation comprises MVT-100+1% D (+) trehalose dihydrate subjected to the proprietary chilling/pressurization process described above to form nanodroplets. The patient received IV administration of nanodroplets (4 mL over a 30-minute infusion period during simultaneous ultrasound. The ultrasound protocol used is as described by Mathias (Mathias, Wilson, et al. 2016 J. Am. Coll. Cardiol. 67.21: 2506-2515). Image-guided diagnostic high mechanical index ultrasound is applied (1.8 MHz; 1.1 to 1.3 mechanical index; 3-ms pulse duration) impulses are applied in the apical 4-, 2-, and 3-chamber views that contained the risk area in the myocardium. Following sonothrombolysis the patient is treated with conventional angioplasty and stenting. Improved myocardial flow is attained and improved left ventricular ejection fraction at 30 days post treatment.

### Example 9

Another patient with acute STEMI is treated with fibrin targeted nanodroplets using similar ultrasound parameters as described in Example 1. It appears that coronary revascularization is attained more rapidly with the targeted nanodroplets than with the untargeted nanodroplets.

### Example 10

A patient with acute ischemic stroke receives IV infusion of 3 vials of fibrin targeted nanodroplets (6 mL total) over a 60-minute period during concomitant IV infusion of t-PA. Ultrasound is applied across the temporal window with a 1 MHz probe at MU=1.0 for the same duration as the simultaneous infusion of t-PA and nanodroplets. Blood flow is rapidly restored to the middle cerebral artery.

### Example 11

A patient has extensive plaque in the left anterior descending coronary artery resulting in a 90% occlusion of the LAD. The patient receives IV infusion of 6 mL of VCAM-1 targeted nanodroplets while ultrasound is applied as in Example 1. This results in diminution of the plaque and improvement in coronary artery blood flow.

### Example 12

A patient has acute peripheral arterial occlusion in the lower extremity. Clot is localized to the femoral artery resulting in loss of blood flow to the leg. An IV infusion is commenced of fibrin targeted nanodroplets. Ultrasound is applied transcutaneously to the region of arterial occlusion using a 3-D ultrasound transducer with center frequency=2 MHz, pulsing the ultrasound 2 seconds on 2 seconds off applying power at 1.6 Megapascals while the nanodroplets are infused IV at a rate of 2.0 cc per hour for two hours. The arterial blockage is removed, and blood flow is restored to the lower extremity.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Unless specifically stated or obvious from context, as used herein, the term “or” is understood to be inclusive.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. An aqueous emulsion or suspension of microbubbles and/or nanodroplets having one or more fibrin-binding ligands attached thereto.

2. The aqueous emulsion or suspension of claim 1, wherein each of microbubbles and/or nanodroplets is conjugated to a plurality of the fibrin-binding ligands.

3. The aqueous emulsion or suspension of claim 1, wherein substantially all of microbubbles and/or nanodroplets is conjugated to a plurality of the fibrin-binding ligands.

4. The aqueous emulsion or suspension of claim 1, wherein the one or more fibrin-binding ligands are fibrin-binding peptides having from about 11 to about 16 amino acids.

5. The aqueous emulsion or suspension of claim 4, wherein the fibrin-binding peptides are selected from Table 1.

6. The aqueous emulsion or suspension of claim 1, wherein the fibrin-binding ligands are conjugated to the microbubbles and/or nanodroplets via a polyethylene glycol (PEG) linker.

7. The aqueous emulsion or suspension of claim 6, wherein the PEG linker has a number average molecular weight (MW) in the rage from about 1,000 to about 10,000 Daltons.

8. The aqueous emulsion or suspension of claim 1, wherein the microbubbles and/or nanodroplets are filled with a gaseous material.

9. The aqueous emulsion or suspension of claim 1, wherein the gaseous material comprises a fluorinated gas.

10. The aqueous emulsion or suspension of claim 9, wherein the fluorinated gas is selected from perfluoromethane, perfluoroethane, perfluoropropane, perfluorocyclopropane, perfluorobutane, perfluorocyclobutane, perfluoropentane, perfluorocylcopentane, perfluorohexane, perfluorocyclohexane, and mixtures of two or more thereof.

11. The aqueous emulsion or suspension of claim 10, wherein the fluorinated gas comprises octafluoropropane.

12. The aqueous emulsion or suspension of claim 1, further comprising a stabilizing agent.

13. The aqueous emulsion or suspension of claim 12, wherein the stabilizing agent is selected from the group consisting of trehalose and D (+) trehalose dihydrate.

14. An aqueous emulsion or suspension of microbubbles and/or nanodroplets having one or more VCAM-1-binding ligands attached thereto.

15-33. (canceled)

34. A method for detecting a vascular thrombus or plaque, comprising: administering to a subject in need thereof an aqueous emulsion or suspension of claim 1; and imaging a part of the subject to detect the presence of vascular thrombus or plaque.

35. A method for diagnosing or assessing thrombosis or atherosclerosis, comprising: administering to a subject in need thereof an aqueous emulsion or suspension of claim 1; and imaging a part of the subject to diagnose or assess thrombosis in the subject.

36. A method for disrupting or destroying vascular thromboses or plaques, comprising: administering to a subject in need thereof an aqueous emulsion or suspension of claim 1; and applying ultrasound to a targeted region of an organ of the subject having vascular thromboses or plaques thereby destroying or reducing the vascular thromboses or plaques.

37. A method for treating thrombosis, atherosclerosis or arterial plaque, comprising: administering to a subject in need thereof an aqueous emulsion or suspension of claim 1; and applying ultrasound to a targeted region of the subject.

38. A method for performing sonothrombolysis, comprising: administering to a subject in need thereof an aqueous emulsion or suspension of claim 1; and applying ultrasound to a targeted region of the subject.

39-42. (canceled)
